(19) United States
(12) Patent Application Publication
(10) Pub. No.: US 2024/0173455 A1
(43) Pub. Date: May 30, 2024

(54) IMAGING APPARATUS, AIR CHANNEL DEVICE AND METHOD FOR OPERATING AN IMAGING APPARATUS

(71) Applicant: Siemens Healthcare GmbH, Erlangen (DE)

(72) Inventors: Martin SEIFERT, Bayreuth (DE); Florian MEISE, Hausen (DE); Philipp HOECHT, Lauf (DE); Lambert FEHER, Stutensee (DE); Bernd MACIEJEWSKI, Markt Bibart (DE)

(73) Assignee: Siemens Healthcare GmbH, Erlangen (DE)

(21) Appl. No.: 18/519,202

(22) Filed: Nov. 27, 2023

(30) Foreign Application Priority Data
Nov. 30, 2022 (EP) 22210431.7

(51) Int. Cl. A61L 9/22

(52) U.S. Cl. CPC A61L 9/22; A61L 2209/14

(57) ABSTRACT

An imaging apparatus has a tubular receptacle to receive an object to be examined by the imaging apparatus. The imaging apparatus has an air channel device configured to: draw air from the tubular receptacle at an intake device of the air channel device; guide drawn-in air through an air filter device of the air channel device for decontamination; and output decontaminated air into the tubular receptacle at an output device of the air channel device. The air channel device is configured to guide a main airstream of air along the tubular receptacle from the output device to the intake device. The main airstream flows through the tubular receptacle towards a receptacle opening of the tubular receptacle.

### CROSS-REFERENCE TO RELATED APPLICATION(S)

The present application claims priority under 35 U.S.C. § 119 to European Patent Application No. 22210431.7, filed Nov. 30, 2022, the entire contents of which is incorporated herein by reference.

### FIELD

One or more example embodiments of the present invention relate to an imaging apparatus, an air channel device and a method for operating an imaging apparatus.

### BACKGROUND

Independent of the grammatical term usage, individuals with male, female or other gender identities are included within the term.

Infection prevention in clinical environments is based on two main pillars, namely hand hygiene (disinfection) and the treatment of relevant surfaces (surface hygiene). As a result of cleaning and disinfecting hands and relevant areas, potential or visible contamination is removed and the risk of infection is reduced. Droplet infection plays a crucial role in the transmission of infective diseases that are based on viruses (e.g. influenza, chickenpox or measles). In this case, the pathogen found in the pharynx or in the respiratory tracts is released into the air via tiny saliva droplets when sneezing, coughing or speaking. These aerosols are breathed in by other people or picked up directly via the mucous membranes of the upper respiratory tract.

Aerosols generally comprise solid or liquid particles of various sizes. A large portion comprises those in the 100-μm range. A portion of significantly smaller particles, smaller than 5 μm or smaller than 2.5 μm, can however also be assumed. Source: DGUV Regulation 102-001, September 2019, in “Regeln für Sicherheit und Gesundheit bei Tätigkeiten mit Biostoffen im Unterricht” [Regulations for Safety and Health when Working with Biochemical Agents in Teaching] published by the Deutsche Gesetzliche Unfallversicherung e.V. DGUV [German Social Accident Insurance]. While droplets of 100 μm diameter require approximately 6 seconds to fall to the ground from a height of 2 m, droplets of 10 μm diameter require 10 minutes for the same distance, and droplets of 1 μm require 16.6 hours. Source: Kappstein, Ines, “Nosokomiale Infektionen: Prävention, Labordiagnostik, antimikrobielle Therapie” [nosocomial infections: prevention, laboratory diagnosis, antimicrobial therapy], 122 tables, Germany, Thieme, 2009. Therefore smaller particles remain in the air for longer, and can disperse in space due to movement of air and influence the occurrence of infection.

Therefore smaller particles remain in the air for longer and can disperse in space due to the movement of air.

The COVID-19 pandemic brought the topic of droplet infection back into public focus and showed that not only those with weak immunity are at risk from such infectious diseases, particularly if the illness is unknown or is not known in advance. The pandemic also showed how effectively the transmission can be reduced by appropriate preventive measures.

With regard to certain imaging methods, an increased risk of droplet infection can occur during the examination of a patient in a restricted examination space, e.g. the tubular receptacle of a magnetic resonance tomography system. The following problems can occur during said examination:
1. Miniscule droplets can build up and remain suspended in the tubular receptacle as a result of natural convection.
2. Suspended miniscule droplet aerosols can be transported, via convective airstreams or thermal natural convection, out of the tubular receptacle and into the examination space in which the imaging apparatus is situated.
3. Larger aerosol droplets can contaminate the surface in the tubular receptacle and the patient handling system.

Until now, use has been made of suitable hygiene and protective measures adapted to the infectious disease concerned, e.g. medical mouth and nose protection or FFP2 protection, whole-body suits, etc. The market also offers a range of filter solutions, e.g. based on HEPA (High-Efficiency Particulate Air/Arrestance) filters, UVC-based methods or plasma-based methods, which decontaminate the room air correspondingly. Combinations of such devices can also be found and are referred to as “filter systems” in the following. It is however a prerequisite of such systems that the system performance and the airstream volumes are tailored precisely to the respective application. A specification of the extent to which the air is contaminated by aerosols is also an important factor with regard to the intended decontamination. In order to disinfect, sterilize or clean air, it must be technically treated in an airstream. This airstream is crucially dependent on the geometry. In the case of mechanical filters, the air must be forced through the filter 4 to 16 times under high pressure using considerable energy and resulting in significant noise emission.

If the air that flows through is treated by UV lamps, the systems must be designed with a moderate airstream. Existing ozonizers/ionizers/plasma systems require extensive interaction chambers, likewise restricting the airstream. All of these methods have it in common that they are deployed as central units due to their long cuboid or elongated structural format.

Recently, mobile surface decontamination devices or stationary UVC lamps have also been deployed for the purpose of surface decontamination. A disadvantage of these commercially available solutions is the implementation in the MRT system, the size of the unit, or the space that is required in the building to improve the effectiveness of the decontamination process. The market-standard solutions are based on electromagnetic discharges and a comparatively slow disinfection effect which is achieved using radicals. These solutions can also comprise a UV lamp.

With regard to medical devices, CN203524686U discloses a CT machine with a self-cleaning function. Disclosed is a sterilization unit based on UVC, plasma or ozone for the CT machine. The publication does not disclose any selective measurement, removal by suction, or decontamination and outlet of potentially contaminated air.

It should be noted here that a validated disinfection measure for an area must result in a microbial reduction of 5 log levels (EU) or 6 log levels (USA). The effect on enveloped or non-enveloped viruses must also be proven using suitable methods in the context of the validation. In the case of UVC-based methods, the required dosage output for effective and efficient decontamination of the air is heavily dependent on the wavelength of the UVC source that is used and on the retention time of the room air in the corresponding filter unit. Experience from surface decontamination using UVC shows that long irradiation times are sometimes required, depending on the distance and the dosage output, in order to guarantee a high level of microbial destruction. Disinfection is also difficult to realize in the case of plasma-based devices. The term “decontamination” is therefore used in the explanations above and below.

### SUMMARY

An object of one or more example embodiments of the present invention is to provide a filter device which allows more efficient decontamination of air in a tubular receptacle of an imaging apparatus.

At least this object is achieved by the respective subject matter of the independent claims. Advantageous developments and preferred embodiment variants are specified in the dependent claims.

A first aspect of an embodiment of the present invention relates to an imaging apparatus having a tubular receptacle for receiving an object that is to be examined by the imaging apparatus, said tubular receptacle having a first tubular receptacle half which faces towards a receptacle opening of the tubular receptacle and a second tubular receptacle half which faces away from the receptacle opening. The imaging apparatus can be e.g. an imaging apparatus for performing computed tomography or for performing magnetic resonance tomography. The receptacle opening can be designed as a tunnel which can be at least partially enclosed by the imaging apparatus. The tubular receptacle can be provided for the purpose of receiving a patient or an object that is to be examined. The tubular receptacle can have the receptacle opening via which the patient or the object that is to be examined can be moved into the tubular receptacle.

The examination object can be situated in the tubular receptacle during the examination by the imaging apparatus, and thus disposed in a recording region of the imaging apparatus.

Provision is made for the imaging apparatus to have an air channel device which is configured to draw air from the tubular receptacle at an intake device of the air channel device, to guide the drawn-in air through an air filter device of the air channel device for the purpose of decontamination, and to output the decontaminated air into the tubular receptacle at an output device of the air channel device. In other words, the imaging apparatus comprises the air channel device which is configured to effect an air exchange in the tubular receptacle. In order to effect the air exchange, the air channel device has the intake device which is configured to supply the air from the tubular receptacle to the air channel device. The intake device can comprise e.g. openings or intake tubes, which can be arranged on a wall of the tubular receptacle. The intake device can have an element for generating an underpressure, e.g. a ventilator, whereby air from the tubular receptacle can be routed into the air channel device. The air channel device has the air filter device, this being designed to decontaminate the air which has been drawn in. The air channel device is configured to guide the air which has been drawn in from the tubular receptacle through the air filter device in order to allow the decontamination of the air by the air filter device. Said decontamination can comprise inactivation of microbes and/or filtering out of aerosols from the drawn-in air by the air filter device. The air filter device can be configured as a mechanical, electrostatic, preferably plasma-based and/or UV-based filter device. The air channel device has an output device which is configured to release the contaminated air from the air channel device into the tubular receptacle. The output device can comprise e.g. openings and/or nozzles on the wall of the tubular receptacle, at which the air is routed from the air channel device into the tubular receptacle after the decontamination.

The air channel device is configured to guide a main airstream of the air along the tubular receptacle from the output device to the intake device, the main airstream running through the tubular receptacle towards a receptacle opening of the tubular receptacle. In other words, the air channel device is configured to provide a main airstream of the air within the tubular receptacle. The main airstream of the air runs within the tubular receptacle from the output device to the intake device. The main airstream is guided via the air channel device in a direction that is oriented towards the receptacle opening of the tubular receptacle. For example, relative to a longitudinal direction of the tubular receptacle, provision can be made for the intake device to be arranged between the output device and the receptacle opening of the tubular receptacle. The air that is output from the output device is consequently drawn in by the intake device, thereby generating the main airstream proceeding from the output device in the direction of the receptacle opening. The main airstream runs into the intake device, such that the main airstream is not routed through the receptacle opening and out of the tubular receptacle.

One or more embodiments of the present invention offer at least the advantage that the main airstream in the tubular receptacle is generated by the air channel device and can provide a patient situated in the tubular receptacle with decontaminated air. At the same time, the intake device prevents contaminated air of the main stream from escaping from the tubular receptacle and thereby contaminating a room in which the imaging apparatus may be located.

One or more embodiments of the present invention also comprise developments which offer further advantages.

According to a development of an embodiment of the present invention, the air channel device has an air barrier device which is configured to output at least a portion of the decontaminated air for the purpose of generating an air barrier in the tubular receptacle between the intake device and the receptacle opening. In other words, provision is made such that the air channel device is configured to output the air that has been decontaminated by the filter device not only via the output device into the tubular receptacle for the purpose of providing the main airstream, but to output a portion of the decontaminated air into the tubular receptacle via the air barrier device. The air barrier device is configured to provide the air barrier in the tubular receptacle by outputting the air. The air barrier device can be configured to output the decontaminated air that is to be output by the air barrier device in one or more predetermined directions at predetermined speeds into the tubular receptacle, said directions and speeds being parameterized in such a way that the air barrier is formed by the air that is output. The air can provide a barrier flow by which the effect of the air barrier is achieved. The barrier flow can run from the air barrier device to the intake device within the tubular receptacle. The air barrier device can be arranged e.g. on an opposite side of the tubular receptacle to the intake device. The barrier flow can run transversely to a longitudinal direction of the tubular receptacle.

This development offers the advantage that an exchange of air between the tubular receptacle and the environment through the receptacle opening is reduced or prevented by the air barrier. It is thereby possible to reduce a risk that aerosols present in the air of the tubular receptacle will leave the tubular receptacle at the receptacle opening.

According to a development of an embodiment of the present invention, the output device comprises at least one free-jet nozzle. In other words, the output device comprises one or more free-jet nozzles for the purpose of outputting the decontaminated air into the tubular receptacle. The free-jet nozzle is configured to output the decontaminated air in a predetermined jet direction. Provision can be made for the output device to protrude into the tubular receptacle, for example. The free-jet nozzle can be configured to output the jet in the longitudinal direction of the tubular receptacle. In this way, the decontaminated air can already be output in the main stream direction during output, for example.

According to a development of an embodiment of the present invention, the air barrier device is configured to output the portion of decontaminated air for providing the air barrier in the tubular receptacle at a guide rail device of the imaging apparatus, said guide rail device being arranged in the tubular receptacle. In other words, the imaging apparatus has the guide rail device which is configured to guide a support for an examination object or a patient into the tubular receptacle. The guide rail device can comprise two guide rails, for example, which can be arranged at a lower side of the tubular receptacle or generally in a lower half of the tubular receptacle and can extend in the longitudinal direction of the tubular receptacle. The air barrier device is configured to output the air for generating the air barrier entirely or at least partly via the guide rail device. For example, the air channel device can be configured to supply the air to the air barrier device, it being possible for the air barrier device to have a channel which leads through the guide rail device to an opening of the air barrier device, said opening being arranged at or in the guide rail device and the air for generating the air barrier being output into the tubular receptacle at said opening.

According to a development of an embodiment of the present invention, the air channel device is configured to guide the main airstream within an upper half of the tubular receptacle. In other words, provision is made for the main airstream to follow a course which runs above a horizontal plane that is situated in a middle of the tubular receptacle.

According to a development of an embodiment of the present invention, the air channel device is configured to guide the main airstream within an upper half of the tubular receptacle. In other words, provision is made for the main airstream to follow a course which runs above a horizontal plane that is situated in a middle of the tubular receptacle. This offers the advantage that the airstream can be guided over a patient.

According to a development of an embodiment of the present invention, the air channel device is configured to adjust an output of the decontaminated air at the output device and the intake of the air at the intake device in such a way that the main airstream through the tubular receptacle has a laminar flow characteristic. In other words, the air channel device is configured both to set the output of the decontaminated air at the output device and to set the intake of the air at the intake device. The air channel device is configured to adjust the output and the intake of the air in such a way that the main airstream has the laminar flow characteristic. The laminar flow characteristic describes a principally laminar flow of the air along the main airstream. The laminar flow characteristic relates to the primary flow pattern, exclusive of unavoidable turbulence in peripheral regions. For example, provision can be made for a control unit of the air channel device to store values for output and intake speeds which are required for the main airstream to exhibit the laminar flow characteristic. The values can be determined beforehand in a simulation of a flow pattern, for example. This development offers the advantage that turbulence of the air can be prevented.

According to a development of an embodiment of the present invention, the air channel device has a further intake device, this being configured to draw air from an environment of the imaging apparatus, and the air channel device is configured to supply the drawn-in air from the environment for decontamination by the air filter device of the air channel device. In other words, the air channel device is configured to draw the air from the environment of the imaging apparatus at the further intake device. This development offers the advantage that e.g. air from the environment can be decontaminated or additional air can be drawn in for provision to the air barrier.

According to a development of an embodiment of the present invention, the air channel device has a further output device, this being configured to output at least a portion of the decontaminated air into an environment of the imaging apparatus. In other words, the air channel device is configured to output at least a portion of the decontaminated air into the environment of the imaging apparatus at the further output device.

According to a development of an embodiment of the present invention, the air filter device is configured as a plasma filter device. In other words, the air filter device is configured to generate a plasma for the purpose of decontaminating the air that is guided through. In this case, the plasma can be provided for the purpose emitting UVC rays by which the air is decontaminated.

According to a development of an embodiment of the present invention, the air filter device has at least one electrode device. The plasma filter device is a filter device for filtering a gas, e.g. air, a plasma being generated for the purpose of filtering the gas. The generated plasma emits UV radiation by which e.g. aerosols or microbes in the gas can be inactivated, destroyed, or particles deactivated.

Provision is made for the electrode device to have a first planar composite electrode and a second planar composite electrode. The composite electrodes can be arranged e.g. in antisymmetrical electromagnetic potential contour. In other words, the electrode device comprises the first composite electrode and the second composite electrode. The composite electrodes of the electrode device can be configured as panel elements in particular. Provision is made for the composite electrodes of the electrode device to be arranged in a reciprocally coplanar manner in a principal surface plane of the electrode device, and physically separated from each other by a discharge gap. In other words, the composite electrodes of the electrode device are situated in the principal surface plane of the electrode device. A discharge gap is situated between the composite electrodes of the electrode device. The discharge gap is a gap in the electrode device, said gap being formed by the composite electrodes in order to allow the passage of the gases to be filtered. Provision is made for each of the composite electrodes to have a respective electrode plate with a respective dielectric coating, at least at a boundary surface of the respective electrode plate relative to the discharge gap. In other words, the electrodes are provided as a composite comprising the respective electrode plate and a dielectric coating on the respective electrode plate. The dielectric coating is deposited on the electrode plate at least at a boundary surface of the respective composite electrode, said boundary surface adjoining the discharge gap.

The filter device has a voltage source which is configured to supply an alternating voltage to the electrode device, said alternating voltage being parameterized to cause the formation of a plasma via a dielectric barrier discharge in the discharge gap. In other words, provision is made for the filter device to comprise the voltage source. The voltage source is provided for the purpose of supplying the alternating voltage to the electrode device in order to cause the formation of the plasma in the discharge gap.

The filter device is configured to guide a gas along a filter flow direction, this being oriented parallel to a normal of the principal surface plane of the electrode device, and through the discharge gap. In other words, the filter device has flow-compatible guide elements, e.g. tube elements, which are configured to influence or define a filter flow direction of the gas in such a way that the gas is guided parallel to the normal of the principal surface plane and through the discharge gap.

In this way, the filter flow direction runs parallel to the normal of the principal surface plane of the electrode device. In other words, the principal flow direction runs perpendicular to the electrode device. Therefore the gas flows perpendicularly through the electrode device and through the discharge gap. As a result of the plasma that is generated in the discharge gap by the dielectric discharge, decontamination of the gas takes place in the region of the discharge gap. The decontamination can be obtained via UVC rays which can be emitted by the plasma and/or by ozone which can form in the discharge gap if the gas is air.

This development offers the advantage that the discharge gap provides a particle trap in which particles remain for longer than molecules of the gas itself. As a result of the particles remaining for longer in the region of the discharge gap, these are exposed to plasma effects such as radicals and UVC for a longer period, thereby increasing the probability of particle inactivation. (Example: a UVC power of 100 W/m2 and a retention time of only 1 s produces a dosage output of 100 J/m2. 90% of coronaviruses are already completely inactivated with effect from 37 J/m2).

According to a development of an embodiment of the present invention, the air filter device is arranged in an electromagnetic screening tube and/or an electromagnetic screening container. In other words, the air filter device is enclosed at least partially by a screening tube and/or a screening container in order to screen electromagnetic radiation and static fields. This development offers the advantage that electromagnetic radiation which can be emitted by the air filter device does not adversely affect the measurements of the imaging apparatus.

A second aspect of an embodiment of the present invention relates to an air channel device for an imaging apparatus. The air channel device is configured to draw air from a tubular receptacle at an intake device of the air channel device, to guide the drawn-in air through an air filter device of the air channel device for the purpose of decontamination, and to output the decontaminated air into the tubular receptacle at an output device of the air channel device. The air channel device can be provided as an integrated part of the imaging apparatus or as an upgrade element for existing imaging apparatuses.

Further embodiment variants of the inventive air channel device follow from the various embodiment variants of the inventive imaging apparatus.

A third aspect of an embodiment of the present invention relates to a method for operating an imaging apparatus, this comprising a tubular receptacle for receiving an object that is to be examined by the imaging apparatus. The method comprises drawing air from the tubular receptacle via an intake device of the air channel device, guiding the drawn-in air through an air filter device of the air channel device in order to decontaminate the air via the air channel device, and outputting the decontaminated air at an output device of the air channel device.

Provision is made for a main airstream of the air to be guided along the tubular receptacle from the output device to the intake device via the air channel device, the main airstream running through the tubular receptacle towards a receptacle opening of the tubular receptacle.

Further embodiment variants of the inventive method follow from the various embodiment variants of the inventive imaging apparatus and the inventive air channel device.

For application cases or application scenarios which could arise in the context of the method and are not explicitly described here, provision can be made for the method to include the output of an error report and/or a request for the input of a user response, and/or the selection of a standard setting and/or a predetermined initial state.

Further features of embodiments of the present invention are derived from the claims, the figures and the description of the figures. The features and combinations of features cited above in the description, and the features and combinations of features cited below in the description of the figures and/or shown in the figures, can be included in embodiments of the present invention not only in the combination that is specified in each case but also in other combinations. In particular, embodiments and combinations of features which do not have all the features in a claim as originally formulated can also be included in the present invention. Furthermore, embodiments and combinations of features which go beyond or vary from the combinations of features that are stated in the back references of the claims can also be included in the present invention.

### BRIEF DESCRIPTION OF THE DRAWINGS

The present invention is explained in greater detail below with reference to specific exemplary embodiments and associated schematic drawings. Identical or functionally identical elements in the figures can be denoted by the same reference signs. The description of identical or functionally identical elements may not necessarily be repeated in relation to various figures.

FIG. 1 shows a schematic illustration of an imaging apparatus;

FIG. 2 shows a schematic illustration of a tubular receptacle with a guide rail device;

FIG. 3 shows a schematic illustration of a flow speed along the tubular receptacle;

FIG. 4 shows a schematic illustration of a flow speed along the tubular receptacle when an air barrier is provided; and

FIG. 5 shows a schematic illustration of a filter device.

### DETAILED DESCRIPTION

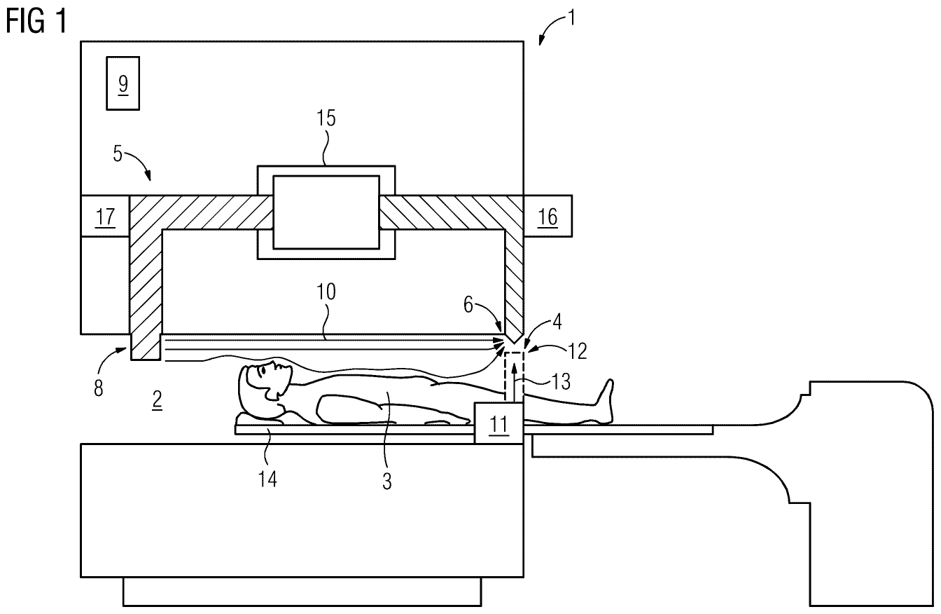

FIG. 1 shows a schematic illustration of an imaging apparatus 1.

The imaging apparatus 1 can be an imaging apparatus 1 for computer tomography or magnetic resonance tomography, for example. The imaging apparatus 1 can have a tubular receptacle 2, which can be provided for receiving an object 3 that is to be examined. For the purpose of examining the object 3, it may be necessary to move the examination object 3 into the tubular receptacle 2 through a receptacle opening 4 of the tubular receptacle 2. The object 3 can be a patient, for example.

As a result of the patient remaining in the tubular receptacle 2, it may be necessary to provide the patient with air. The imaging apparatus 1 can have an air channel device 5 for this purpose. In this case, it may be necessary to decontaminate contaminated air which is released by the patient, for example, in order to avoid an adverse effect on the patient and/or an accumulation of microbes on a surface of the tubular receptacle 2. At the same time, it may be necessary to prevent an escape of the contaminated air from the receptacle opening 4 of the tubular receptacle 2 into an environment of the imaging apparatus 1.

For this purpose, provision can be made for the air channel device 5 to have an intake device 6 which can be configured to draw air from the tubular receptacle 2. The air can contain aerosols, for example, such that it can be necessary to guide the air through an air filter device 7 of the air channel device 5 for the purpose of decontamination. The intake device 6 can preferably be arranged in the vicinity of the receptacle opening 4 of the tubular receptacle 2, in order to prevent the escape of contaminated air from the tubular receptacle 2. The intake device 6 can be provided in particular for placement in an upper half of the tubular receptacle 2. The air filter device 7 can be configured as a plasma filter, for example, which is configured to decontaminate the contaminated air by UV radiation generated by the plasma 28. The air channel device 5 can be configured to supply the decontaminated air emerging from air filter device 7 to an output device 8 in order to output the decontaminated air into the tubular receptacle 2.

The output device 8 can be arranged at an end of the tubular receptacle 2 which is opposite to the receptacle opening 4, so that the decontaminated air is output in a region of the tubular receptacle 2 in which the face of the patient is usually situated. The intake device 6 and the output device 8 can be arranged in such a way that a main airstream 10 of the air runs through the tubular receptacle 2 from the output device 8 to the intake device 6 in the direction of the receptacle opening 4 of the tubular receptacle 2. The main airstream 10 can run in an upper half of the tubular receptacle 2 in particular. The air channel device 5 can have a control unit 9 which is able to control the intake device 6, the air filter device 7 and the output device 8 in order to influence a volume of an airstream. This can allow the output device 8 and the intake device 6 to be controlled in such a way that the air running along the main airstream 10 has a laminar flow characteristic. For this purpose, the control unit 9 can store control values which are then set in the output device 8 and the intake device 6 in order to allow the laminar flow characteristic of the main stream. The intake device 6 can be arranged between the receptacle opening 4 and the output device 8 relative to a longitudinal direction of the tubular receptacle 2, in order to prevent the air of the main stream from escaping via the receptacle opening 4.

The air channel device 5 can have an air barrier device 11 which can be configured to generate an air barrier 12 between the intake device 6 and the receptacle opening 4. It is thereby possible to prevent the air from escaping via the receptacle opening 4. For the purpose of producing the air barrier 12, provision can be made for at least a portion of the decontaminated air to be output in a predetermined direction by the air barrier device 11 in order to generate a predetermined barrier flow 13. The barrier flow 13 can bring about a circulation of air, which can result in the formation of the air barrier 12. The air barrier device 11 can be configured to output the barrier flow 13 via a guide rail device 14 of the imaging apparatus 1.

Provision can be made for the air channel device 5 to have a further intake device 16, which can be configured to draw air from the environment of the imaging apparatus 1. The air that is drawn from the environment can likewise be guided by the air channel device 5 through the air filter device 7 for the purpose of decontamination. It is thereby possible to provide for additional air to be drawn from the environment, e.g. in order to provide the barrier flow 13. The air channel device 5 can have a further output device 17 via which a portion of the decontaminated air can be supplied to the environment.

The air channel device 5 can have a screening container 15 which at least partially encloses the air filter device 7 in order to prevent an adverse effect on the measurement of the imaging apparatus 1 as a result of electromagnetic radiation that may be output by the air filter device 7.

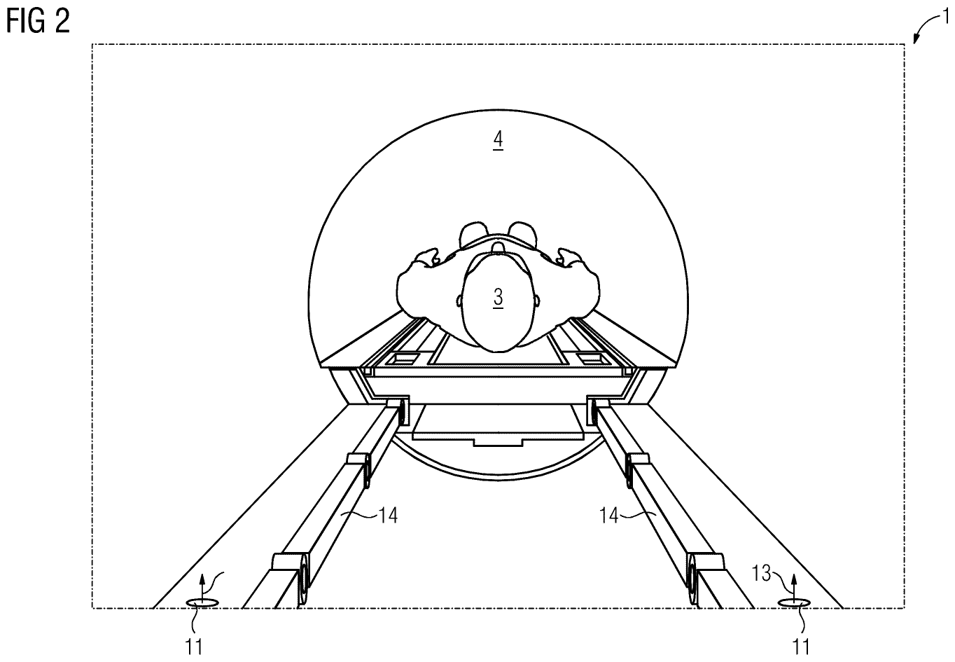

FIG. 2 shows a schematic illustration of a tubular receptacle 2 with a guide rail device 14.

For example, provision can be made for the air barrier device 11 to comprise two openings though which a portion of the decontaminated air can be output in predetermined directions in order to form the barrier flow 13. The barrier flow 13 can be guided in a transverse direction of the tubular receptacle 2, for example, in order to form the air barrier 12 in the transverse direction of the tubular receptacle 2.

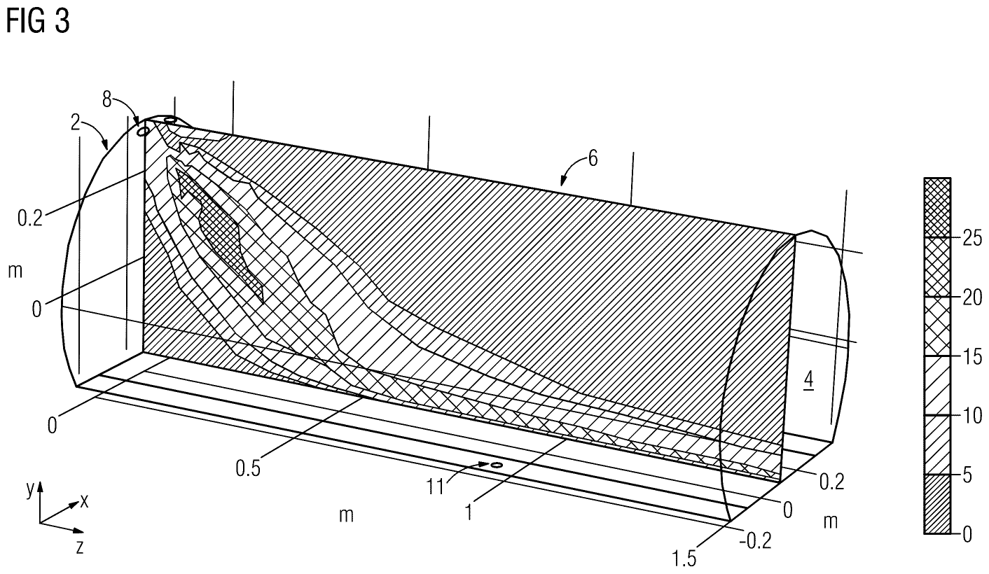

FIG. 3 shows a schematic illustration of a flow speed along the tubular receptacle 2.

Shown is a region of raised speed of the air coming from the output device 8 of the air channel device 5 in the tubular receptacle 2.

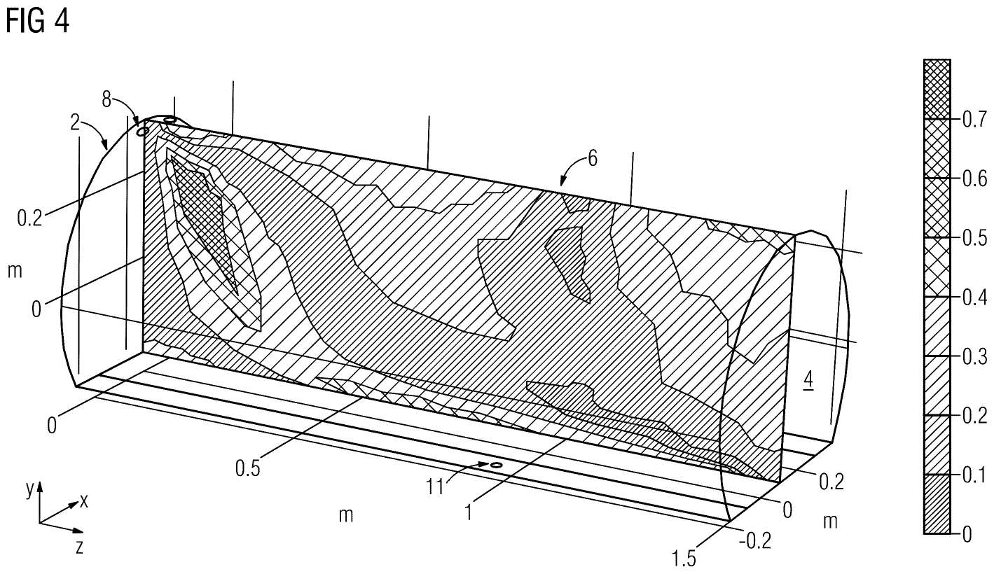

FIG. 4 shows a schematic illustration of a flow speed along the tubular receptacle 2 when an air barrier 12 is provided.

FIG. 4 shows the simulation results for the air circulation in the tubular receptacle 2 as a result of the air barrier 12. It can be seen that this arrangement prevents contaminated air from being transported out of the tubular receptacle 2.

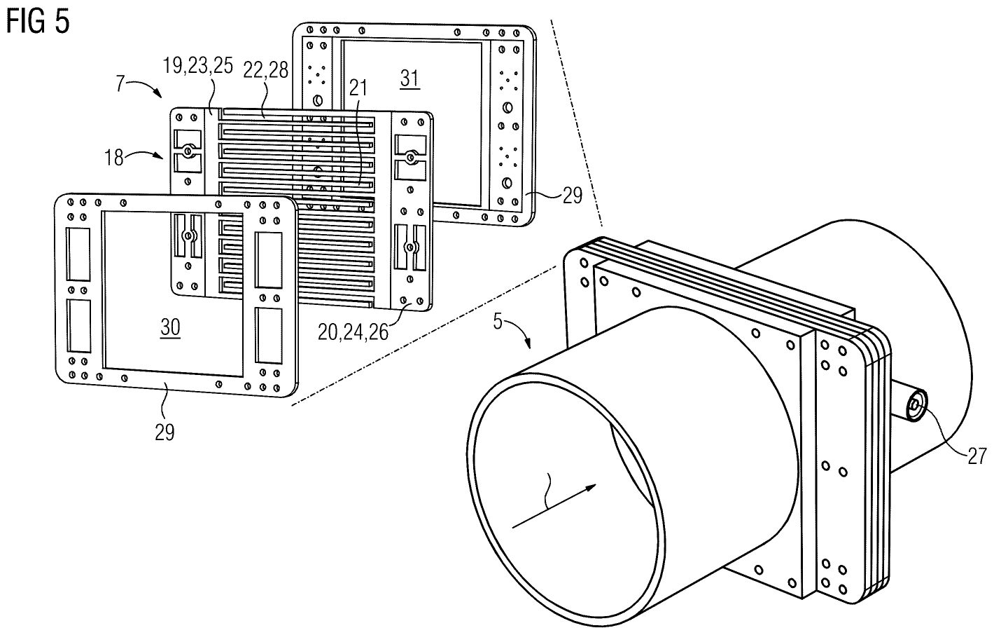

FIG. 5 shows a schematic illustration of an air filter device 7.

The air filter device 7 can have a holding device 29 which can be provided for the purpose of arranging the electrode devices 18, the dust filter 30 and the active carbon filter 31 in predetermined positions. A feed-in point can be arranged on the holding device, and can be provided for the purpose of supplying the electrode devices 18 with the alternating voltage from the voltage source 27. In order to effect a passage of the air in the filter flow direction 32, one or two tubes can be arranged on the air filter device 7, said tubes being arranged on the holding device 29 possibly via a flange.

The electrode device 18 of the air filter device 7 can have a first composite electrode 19 and a second composite electrode 20. The first composite electrode 19 and the second composite electrode 20 can be planar and lie in a principal surface plane 21 of the electrode device 18. The first composite electrode 19 and the second composite electrode 20 can be arranged in a reciprocally coplanar manner, for example. The composite electrodes 19, 20 can be manufactured from a source plate which can be divided into a first electrode plate 23 and a second electrode plate 24 by producing a discharge gap 22 in the source plate, such that the first electrode plate 23, 24 and the second electrode plate 23, 24 can be separated from each other by the discharge gap 22. The two composite electrodes 19, 20 can each have a respective electrode plate 23, 24 which can be coated with a respective dielectric coating 25, 26, whereby the respective electrodes can be composites. The electrode plate 23, 24 of the respective composite electrode 19, 20 can comprise e.g. aluminum, an aluminum alloy and/or stainless steel as a material. The dielectric coating 25, 26 of the respective composite electrode 19, 20 can be deposited on the respective electrode plate 23, 24 at least at a boundary surface of the respective composite electrode 19, 20 relative to the discharge gap 22. The function of the dielectric coating 25, 26 can consist in allowing a dielectric discharge in the discharge gap 22 when a correspondingly parameterized electric alternating voltage is applied to the electrode device 18. The dielectric coating 25, 26 can comprise one or more polymers as a material. The possible polymers can comprise e.g. fluoropolymers, in particular polyvinylidene fluoride and/or polytetrafluoroethylene. Graphite fluoride can also be admixed to the at least one polymer. The dielectric coating 25, 26 can also comprise one or more ceramics as a material, in particular barium titanate.

In the following description, the term air filter device 7 is used for a structure which comprises a filter unit, a power supply, a screen for screening electromagnetic radiation and all relevant cables and other interfaces. The air filter device 7 can also be referred to as a decontamination device.

The air filter device 7 can be provided for arrangement in an air channel device 5 for the purpose of cleaning air/aerosols in the tubular receptacle 2 of the imaging apparatus 1.

Provision can be made for generating a predetermined flow pattern in the tubular receptacle 2 of the imaging apparatus 1 in order to prevent contaminated air from escaping from the tubular receptacle 2 to the outside. The flow pattern can comprise an air barrier 12, also known as an air curtain.

The air filter device 7 can allow a decontamination of air via plasma 28. The air filter device 7 can be arranged in the existing air channel device 5 of an imaging apparatus 1 with a relatively high degree of effectiveness and made-to-measure geometry. Alternatively, the air filter device 7 can be arranged as a separate unit which is adapted to the imaging apparatus 1.

By virtue of the small structural space that is occupied by the air filter device 7, effective measures for ensuring electromagnetic compatibility can be realized.

In order to decontaminate the air inside the tubular receptacle 2, in contrast with known imaging apparatuses 1, the output device 8 can be adapted to the intake device 6. With regard to safety considerations such as the protection of the patient or the operator against helium in the event of leakage or quenching, a further intake device 16 6 can be arranged on the floor of the imaging apparatus 1. The air that is drawn in from the environment by the further intake device 16 can be routed to an output device 8 directly above the patient in the tubular receptacle 2.

The air filter device 7 can be placed at any suitable position in the air channel device 5. The standard dimensions of the tubes used in the air channel device 5 can have a diameter of 55 mm, for example. The air flow rates can lie in the range 250 to 300 m³/h, for example. Simulation results based on these requirements show that a minimum size of 84×56×1 mm could be suitable for an electrode of the air filter device 7. By virtue of this very small structural size, effective measures can be realized for electromagnetic compatibility in order to ensure compatibility with the HF field and the B field when the air filter device 7 is used in an imaging apparatus 1. This can be achieved by installing the air filter device 7 and a plasma stream generator of the air filter device 7 in a metal tube. The metal tube can comprise special steel, for example.

In order to ensure that contaminated air is guided through the air filter device 7, an intake device 6 can be attached at the receptacle opening 4 of the tubular receptacle 2. In respect of the airstream volume within the tubular receptacle 2 and the laminar airstream above the patient, this intake device 6 can be set in such a way that the contaminated air is completely replaced within the tubular receptacle 2.

In order to generate a constant laminar airstream above the patient in the tubular receptacle 2, the output device 8 can comprise made-to-measure free-jet nozzles which can be positioned at an opposite end to the intake device 6 in a longitudinal direction.

A physical isolation of the airstream in the tubular receptacle 2 can be achieved by stopping the main stream that moves towards the receptacle opening 4. To this end, at least a portion of the decontaminated air can be routed into the tubular receptacle 2 via the air barrier device 11. The air barrier device 11 can comprise two air inlets, these being situated on the left-hand and right-hand guiding bars of the guide rail device 14 within the tubular receptacle 2.

By virtue of using the described air filter device 7, it is possible to lengthen the maintenance interval of the filter in comparison with air filter devices 7 according to the prior art, which require replacement annually.

An air filter device 7 based on the principle of the dielectric discharge is an economical, practically maintenance-free, planar, scalable and universally applicable, energy-efficient plasma filter unit for the disinfection of air. This preventive technology is suitable for universal application against air-transmitted infection. The plasma technology is therefore an ideal, energy-efficient improvement over conventional mechanical filters such as HEPA filters, which require maximum differential pressure, and the inactivation of pathogens via UVC radiation, which is universally effective against all pathogens but requires large interaction paths. While plasma-induced radical reactions and UVC radiation inactivate gaseous compounds, odors, micro-organisms and viruses, conventional largely maintenance-free self-sterilizing filter elements effect the mechanical separation of dust particles and aerosols in a technical combination of complementary individual elements.

Central mechanical HEPA filters can be replaced by the compact air filter device 7, which can comprise plasma electrodes. Vast quantities of toxic waste can be avoided thereby. All that remains is a self-sterilizing dust and moisture prefilter at the input and a thin active carbon filter 31 at the output of the plasma electrodes of the air filter device 7. By virtue of the air filter device 7, it is possible to dispense with additional UV lamps and the associated technical means, devices, mechanisms, etc., at the same time as achieving a greater disinfection effect.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. An imaging apparatus comprising:
a tubular receptacle configured to receive an object to be examined by the imaging apparatus; and
an air channel device configured to
draw air from the tubular receptacle at an intake device of the air channel device,
guide drawn-in air through an air filter device of the air channel device for decontamination, and
output decontaminated air into the tubular receptacle at an output device of the air channel device; wherein
the air channel device is configured to guide a main airstream of air along the tubular receptacle from the output device to the intake device, and
the main airstream flows through the tubular receptacle towards a receptacle opening of the tubular receptacle.

2. The imaging apparatus as claimed in claim 1, wherein the output device has at least one free-jet nozzle.

3. The imaging apparatus as claimed in claim 1, wherein the air channel device includes an air barrier device configured to output at least a portion of the decontaminated air to generate an air barrier in the tubular receptacle between the intake device and the receptacle opening.

4. The imaging apparatus as claimed in claim 3, wherein the air barrier device is configured to output the portion of the decontaminated air for generating the air barrier in the tubular receptacle at a guide rail device of the imaging apparatus, said guide rail device being arranged in the tubular receptacle.

5. The imaging apparatus as claimed in claim 1, wherein the air channel device is configured to guide the main airstream within an upper half of the tubular receptacle.

6. The imaging apparatus as claimed in claim 1, wherein the air channel device is configured to adjust the output of the decontaminated air at the output device and an intake of the air at the intake device such that the main airstream through the tubular receptacle has a laminar flow characteristic.

7. The imaging apparatus as claimed in claim 1, wherein the air channel device comprises:
a further intake device configured to draw air from an environment of the imaging apparatus, wherein
the air channel device is configured to supply drawn-in air from the environment for decontamination by the air filter device of the air channel device.

8. The imaging apparatus as claimed in claim 1, wherein the air channel device comprises:
a further output device configured to output at least a portion of the decontaminated air into an environment of the imaging apparatus.

9. The imaging apparatus as claimed in claim 1, wherein the air filter device is configured as a plasma filter.

10. The imaging apparatus as claimed in claim 9, wherein the air filter device comprises:
at least one electrode device, the at least one electrode device having a first planar composite electrode and a second planar composite electrode, wherein
the first planar composite electrode and the second planar composite electrode are arranged in a reciprocally coplanar manner in a principal surface plane of the at least one electrode device, and the first planar composite electrode and the second planar composite electrode are physically separated from each other by a discharge gap, and
each of first planar composite electrode and the second planar composite electrode has a respective electrode plate with a respective dielectric coating at least at a boundary surface of the respective electrode plate relative to the discharge gap; and
a voltage source configured to supply an alternating voltage to the at least one electrode device, wherein
the alternating voltage is parameterized to cause formation of a plasma via a dielectric barrier discharge in the discharge gap, and
the plasma filter is configured to guide the air along a filter flow direction, the filter flow direction being oriented parallel to a normal of the principal surface plane of the at least one electrode device, and through the discharge gap.

11. The imaging apparatus as claimed in claim 1, wherein the air filter device is arranged in at least one of a screening tube or a screening container.

12. An air channel device for an imaging apparatus, said air channel device configured to
draw air from a tubular receptacle at an intake device of the air channel device,
guide drawn-in air through an air filter device of the air channel device for decontamination, and
output decontaminated air into the tubular receptacle at an output device of the air channel device.

13. A method for operating an imaging apparatus having a tubular receptacle configured to receive an object to be examined by the imaging apparatus, the method comprising:
drawing in air from the tubular receptacle via an intake device of an air channel device of the imaging apparatus;
guiding drawn-in air through the air channel device for decontamination by an air filter device of the air channel device; and
outputting decontaminated air into the tubular receptacle via an output device of the air channel device, wherein
a main airstream of air is guided along the tubular receptacle from the output device to the intake device via the air channel device, and
the main airstream flows through the tubular receptacle towards a receptacle opening of the tubular receptacle.

14. The imaging apparatus as claimed in claim 3, wherein the air channel device is configured to adjust the output of the decontaminated air at the output device and an intake of the air at the intake device such that the main airstream through the tubular receptacle has a laminar flow characteristic.

15. The imaging apparatus as claimed in claim 3, wherein the air filter device is configured as a plasma filter.

16. The imaging apparatus as claimed in claim 15, wherein the air filter device comprises:
at least one electrode device, the at least one electrode device having a first planar composite electrode and a second planar composite electrode, wherein
the first planar composite electrode and the second planar composite electrode are arranged in a reciprocally coplanar manner in a principal surface plane of the at least one electrode device, and the first planar composite electrode and the second planar composite electrode are physically separated from each other by a discharge gap, and
each of first planar composite electrode and the second planar composite electrode has a respective electrode plate with a respective dielectric coating at least at a boundary surface of the respective electrode plate relative to the discharge gap; and
a voltage source configured to supply an alternating voltage to the at least one electrode device, wherein
the alternating voltage is parameterized to cause formation of a plasma via a dielectric barrier discharge in the discharge gap, and
the plasma filter is configured to guide the air along a filter flow direction, the filter flow direction being oriented parallel to a normal of the principal surface plane of the at least one electrode device, and through the discharge gap.

17. The imaging apparatus as claimed in claim 4, wherein the air channel device is configured to adjust the output of the decontaminated air at the output device and an intake of the air at the intake device such that the main airstream through the tubular receptacle has a laminar flow characteristic.

18. The imaging apparatus as claimed in claim 4, wherein the air filter device is configured as a plasma filter.

19. The imaging apparatus as claimed in claim 18, wherein the air filter device comprises:
at least one electrode device, the at least one electrode device having a first planar composite electrode and a second planar composite electrode, wherein
the first planar composite electrode and the second planar composite electrode are arranged in a reciprocally coplanar manner in a principal surface plane of the at least one electrode device, and the first planar composite electrode and the second planar composite electrode are physically separated from each other by a discharge gap, and
each of first planar composite electrode and the second planar composite electrode has a respective electrode plate with a respective dielectric coating at least at a boundary surface of the respective electrode plate relative to the discharge gap; and
a voltage source configured to supply an alternating voltage to the at least one electrode device, wherein
the alternating voltage is parameterized to cause formation of a plasma via a dielectric barrier discharge in the discharge gap, and
the plasma filter is configured to guide the air along a filter flow direction, the filter flow direction being oriented parallel to a normal of the principal surface plane of the at least one electrode device, and through the discharge gap.

20. The imaging apparatus as claimed in claim 5, wherein the air channel device is configured to adjust the output of the decontaminated air at the output device and an intake of the air at the intake device such that the main airstream through the tubular receptacle has a laminar flow characteristic.
